(19) United States
(12) Patent Application Publication
(10) Pub. No.: US 2025/0169901 A1
(43) Pub. Date: May 29, 2025

(54) METHOD AND SYSTEM FOR OPTIMIZING ROBOT BASE LOCATION FOR MAXIMIZED ROBOT MANIPULABILITY

(71) Applicant: EDDA TECHNOLOGY, INC., PRINCETON, NJ (US)

(72) Inventors: Yash Evalekar, PRINCETON, NJ (US); Yuanfeng Mao, PRINCETON, NJ (US); Guo-Qing Wei, Plainsboro, NJ (US); Li Fan, Belle Mead, NJ (US); Xiaolan Zeng, Princeton, NJ (US); Jianzhong Qian, Princeton Junction, NJ (US)

(21) Appl. No.: 19/040,245

(22) Filed: Jan. 29, 2025

Related U.S. Application Data
(63) Continuation-in-part of application No. 18/163,665, filed on Feb. 2, 2023.

(51) Int. Cl. A61B 34/30; A61B 34/10; A61B 34/20

(52) U.S. Cl. CPC A61B 34/30; A61B 2034/107; A61B 2034/2065; A61B 2034/302

(57) ABSTRACT

The present teaching relates to determining an optimal robot base location that maximizes robot manipulability. A trocar location is determined for inserting a surgical instrument manipulated by a robot to reach a target organ. With respect to the trocar location, candidate base locations are generated, each of which is a location to deploy the robot for manipulating the surgical instrument through the trocar location. Each candidate base location is evaluated based on criteria indicative of the robot's manipulability of the surgical instrument with respect to the target organ. An optimal base location is selected from the candidate base locations based on the evaluation result.

### CROSS REFERENCE TO RELATED APPLICATIONS

The present application is a continuation in part of U.S. application Ser. No. 18/163,665, filed on Feb. 2, 2023, entitled “SYSTEM AND METHOD FOR AUTOMATED DETERMINATION OF ROBOT BASE LOCATION VIA TROCAR′S ACCESSIBILITY MAP”, which is related to U.S. application Ser. No. 18/163,686, filed on Feb. 2, 2023, entitled “SYSTEM AND METHOD FOR AUTOMATED TROCAR AND ROBOT BASE LOCATION DETERMINATION”, and U.S. application Ser. No. 18/163,703 filed on Feb. 2, 2023, entitled “SYSTEM AND METHOD FOR AUTOMATED SIMULTANEOUS TROCAR AND ROBOT BASE LOCATION DETERMINATION”, the contents of which are hereby incorporated by reference in their entireties.

### BACKGROUND

### 1. Technical Field

The present teaching generally relates to computers. More specifically, the present teaching relates to signal processing.

### 2. Technical Background

Robots in the past few decades have been deployed in different situations, including in industrial settings such as in assembly lines that assemble products 24/7 and in a warehouse for transporting goods or being deployed in a surgery room to assist surgeons in different types of surgical operations. In recent years, robotic surgery has been more widely accepted in, e.g., liver resection operations, due to robots' incomparable precision, reachability, and flexibility in tasks that may be more difficult for humans to do. Additional notable characteristic of a robot is the fact that its performance does not degrade over time as compared with a human who gets tired, needs to eat, and sleep, and can be distracted.

In a robot assisted surgery, a robot may be deployed to work with doctors or nurses to perform certain specified tasks and may be positioned at a certain location in the surgery room to allow the robot to do so. Conventionally, the placement of a robot in a surgery room is done by a human manually based on, e.g., some sense about what location in a surgery room may be suitable for what is to be performed by the robot, a location of a tool to be handled by the robot to perform the task, and the nature of the operation, etc. Such human sense may be vague, imprecise, and even incorrect, which is problematic. In addition, although manual placement decisions may consider the relative locations of different targets, the intrinsic manipulability of the robot is not addressed.

Thus, there is a need to develop solutions that address the shortcomings of the current state of the art.

### SUMMARY

The teachings disclosed herein relate to methods, systems, and programming for information management. More particularly, the present teaching relates to methods, systems, and programming related to content summarization.

In one example, a method is disclosed for determining an optimal robot base location that maximizes robot manipulability. A trocar location is determined for inserting a surgical instrument manipulated by a robot to reach a target organ. With respect to the trocar location, candidate base locations are generated, each of which is a location to deploy the robot for manipulating the surgical instrument through the trocar location. Each candidate base location is evaluated based on criteria indicative of the robot's manipulability of the surgical instrument with respect to the target organ. An optimal base location is selected from the candidate base locations based on the evaluation result.

In a different example, a system is disclosed for determining an optimal robot base location that maximizes robot manipulability. The system includes a trocar insertion location optimizer, a base location generation unit, and an optimal robot base location determiner. The trocar insertion location optimizer is for determining a trocar location for inserting a surgical instrument manipulated by a robot to reach a target organ. The base location generation unit is for generating, with respect to the trocar location, candidate base locations, each of which is a location to deploy the robot for manipulating the surgical instrument through the trocar location. The optimal robot base location determiner evaluates each candidate base location based on criteria indicative of the robot's manipulability of the surgical instrument with respect to the target organ and selects an optimal base location from the candidate base locations based on the evaluation result.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory and tangible medium having information recorded thereon for determining an optimal robot base location that maximizes robot manipulability. A trocar location is determined for inserting a surgical instrument manipulated by a robot to reach a target organ. With respect to the trocar location, candidate base locations are generated, each of which is a location to deploy the robot for manipulating the surgical instrument through the trocar location. Each candidate base location is evaluated based on criteria indicative of the robot's manipulability of the surgical instrument with respect to the target organ. An optimal base location is selected from the candidate base locations based on the evaluation result.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 shows a surgical setting in which a robot is deployed to handle some aspects of a surgery;

FIG. 2 shows a surgical instrument inserted into a patient's body via a trocar point that yields a corresponding access map over an organ to be operated on, in accordance with an embodiment of the present teaching;

FIG. 3A illustrates that different maneuvers are needed to enable a surgical instrument inserted from different trocar locations to reach an area on an organ, in accordance with an embodiment of the present teaching;

FIG. 3B illustrates different considerations in evaluating a candidate trocar point, in accordance with an embodiment of the present teaching;

FIG. 4A depicts a surgical setting in which a robot may be placed at different base locations, in accordance with an embodiment of the present teaching;

FIG. 4B is shows different robot base locations around a surgical table organized as an operating space grid, in accordance with an embodiment of the present teaching;

FIG. 4C shows different considerations in evaluating a robot base location, in accordance with an embodiment of the present teaching;

FIG. 5A provides an exemplary distribution of candidate robot base locations in a surgery room and classifications thereof, in accordance with an embodiment of the present teaching;

FIG. 5B depicts an exemplary high level system diagram of a robot base location optimization scheme based on robot manipulability with respect to given surgical information, in accordance with an embodiment of the present teaching;

FIG. 5C is a flowchart of an exemplary process of a robot base location optimization scheme, in accordance with an embodiment of the present teaching;

FIG. 6A shows exemplary types of surgery information to be considered in determining a robot's manipulability, in accordance with an embodiment of the present teaching;

FIG. 6B illustrates exemplary type of criteria in assessing the optimality of a robot base location given surgery information, in accordance with an embodiment of the present teaching;

FIG. 6C illustrates exemplary spatial relations among a surgery area, a trocar location, and a robot base location, in accordance with an exemplary embodiment of the present teaching;

FIG. 6D illustrates an exemplary surgery area encompassing a target therein, in accordance with an embodiment of the present teaching;

FIG. 7A depicts an exemplary high-level system diagram of an optimal robot base location determiner, in accordance with an embodiment of the present teaching;

FIG. 7B is a flowchart of an exemplary process of an optimal robot base location determiner, in accordance with an embodiment of the present teaching;

FIG. 8A is a flowchart of an exemplary process of assessing reachability/continuity of each desired reachable point in a surgery area, in accordance with an embodiment of the present teaching;

FIG. 8B is a flowchart of an exemplary process of assessing the success rate with respect to each base location, in accordance with an embodiment of the present teaching; and

FIG. 9 is an illustrative diagram of an exemplary computing device architecture that may be used to realize a specialized system implementing the present teaching in accordance with various embodiments.

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well-known methods, procedures, components, and/or systems have been described at a relatively high-level, without detail, in order to avoid unnecessarily obscuring aspects of the present teachings.

The present teaching discloses exemplary methods, systems, and implementations for automatically selecting a robot base location in a surgery room to optimize the robot manipulability to ensure that an surgical instrument manipulated by a robot deployed at a base location can maximize the reach to each point in a surgery area with a target organ enclosed therein. Surgery related information may be provided which may include a target organ, a region around the target organ to be affected by the surgery, and/or a 3D model for the target organ. Such information may be used to determine a trocar location, which is where a surgical instrument is inserted for moving towards the target organ. The surgery information may also define a surgery area SA specified with a center point C and the dimensions of the SA. In some embodiments, the SA may be defined as a cube with a length on each side N as specified in the surgery information. To ensure that the surgical instrument manipulated by the robot from a base location can reach any area in the SA, the surgery information map also include information about how to divide the SA into a grid with non-overlap sub-areas therein, each of which may be of a dimension defined via the surgery information.

With the trocar location identified, candidate base locations in a surgery room may be generated automatically. Each of the candidate base locations may be evaluated in terms of its manipulability of the surgical instrument to maximize its reach to as many as possible the sub-areas in the SA grid. According to the present teaching, the evaluation of each candidate base location may be performed with respect to some criteria, some of which are directed to the coverage of the sub-areas in the SA grid and some may be directed to whether each of the sub-areas may be accessed in a smooth and continuous manner. In some embodiments, with respect to each sub-area in the SA grid, its reachability and continuity may be assessed with respect to each candidate base location. The reachability may be defined as whether the robot at a given candidate base location can manipulate the surgical instrument through the trocar point to reach the sub-area. The continuity associated with a sub-area may be defined as whether the robot can manipulate the surgical instrument through the trocar point to reach the sub-area from all of its neighboring sub-areas.

The objective is that a robot deployed at a base location can manipulate the surgical instrument to reach all sub-areas in the SA grid, including to reach each sub-area from any of its neighboring sub-areas. In this ideal situation, the evaluation result with respect to the base location is a 100% on both the reachability coverage as well as the continuity across all sub-areas. In some embodiments, in addition to the reachability and continuity associated with each sub-area, some metrics indicative of the overall success rate may also be obtained based on the individual reachability/continuity associated with sub-areas. In general, an optimal base location may be selected from multiple candidate base locations that corresponds to one that allows the robot to achieve an optimal performance, defined based on the need of applications. For example, in some applications, it may be more important to have a maximum coverage, i.e., can reach as many sub-areas as possible. In some applications, the continuity may be more important, and, in this case, the optimal base location may be the one that leads to the highest number of sub-areas with continuity.

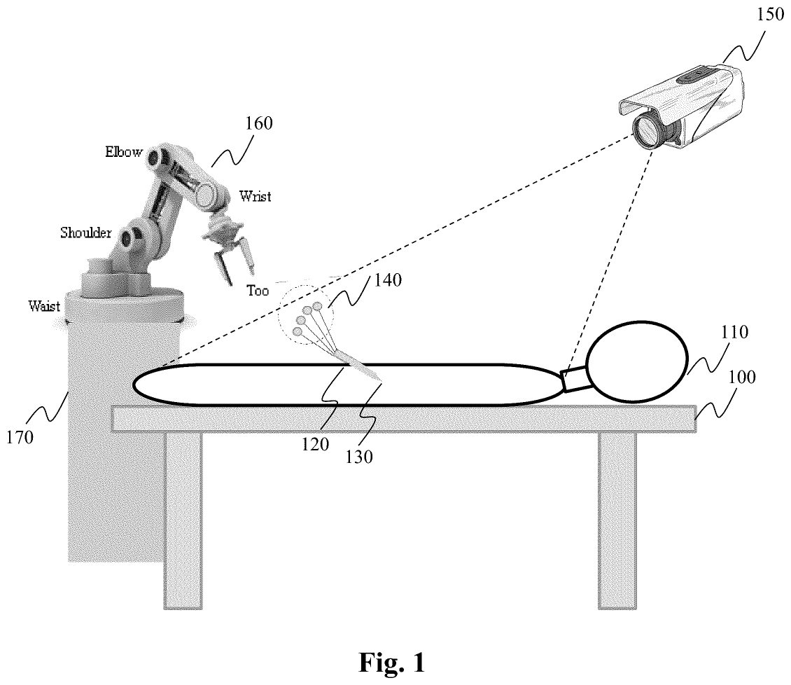

FIG. 1 shows a workspace in a surgical setting in which a robot 160 is deployed to handle some aspects of a surgery. In this workspace, there is a surgical table 100, a patient 110 on the surgical table with a surgical instrument 120 inserted therein having a rigid body and a tip 130. A tracking mechanism is deployed in the workspace with a sensor, e.g., a camera 150, that is configured to monitor tracking devices 140 attached to one end of the surgical instrument 120 that is outside of the patient's body. Assume that the sensor 150 is calibrated with respect to the workspace, i.e., when it observes the tracking devices, the tracking mechanism can determine the 3D coordinate of the surgical instrument in a coordinate system defined with respect to the workspace. As the surgical instrument is a rigid body (including the body 120 and the tip portion 130), through the detection of the tracking devices, the 3D coordinate of the tip 130 of the surgical instrument may also be accordingly determined.

In this exemplary surgical setting, the robot 160 may have a base 170 and the base 170 is moved, the robot may move accordingly to different locations. A surgical robot may be deployed to, e.g., handle some aspects of the operation in a surgery. For instance, robot 160 may be used to manipulate the movement of the surgical instrument 120 in a manner so that the tip 130 of the instrument reaches some specified 3D coordinate in the workspace, e.g., a particular cut point on an organ of the patient. Such manipulation may be achieved by configuring kinematic parameters of the robot in such a way that it can control the instrument to travel along a path from a current location of tip 130 to a specified 3D coordinate. Depending on the base location of robot 160, the robot needs to operate differently to achieve the goal. As discussed herein, depending on the base location, the robot 160 may yield better or more efficient performance.

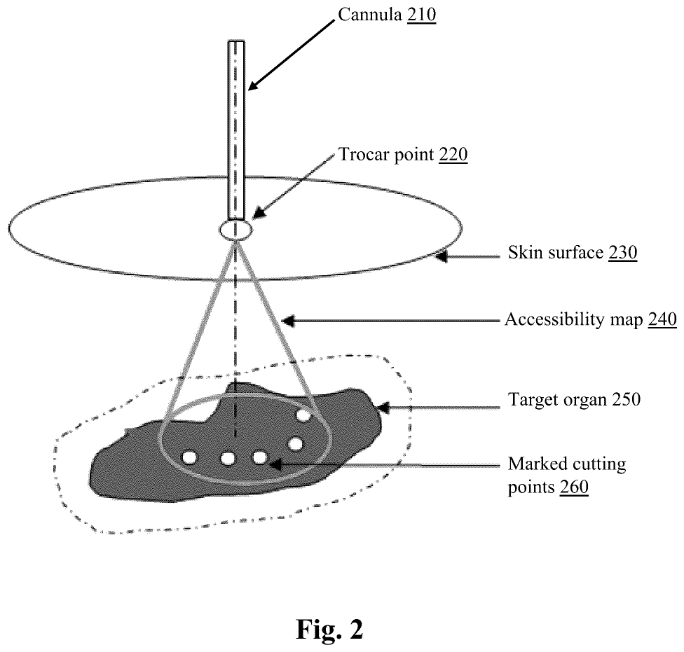

The base location needed for robot 160 to perform a desired function may differ with respect to the insertion location of the surgical instrument at a trocar point on the skin of the patient. The location of the trocar point is usually determined with respect to a target area and/or various target points therein the surgical instrument 120 needs to reach. For example, the surgical instrument 120 may be used to resect a portion of an organ so that the tip of the surgical instrument 120 needs to reach a series of cut points on the organ from the trocar point. FIG. 2 shows a surgical instrument, i.e., a cannula 210, inserted into a patient's skin 230 via a trocar point 220. Through the trocar point 220, the cannula 210 can reach various points in a target organ 250 forming a corresponding access map 240 over the target organ 250. To operate on the target organ, a series of cut points 260 may be preplanned prior to the surgery, as shown in FIG. 2. To ensure that the cannula 210 can reach the preplanned cut points 260, the access map 240 determined with respect to the trocar point 220 needs to cover all cut points 260. That is, a trocar point may be selected in such a way that its corresponding access map encloses all cut points therein.

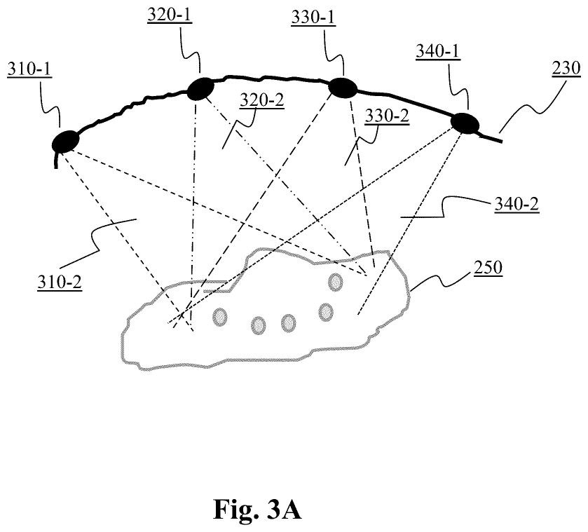

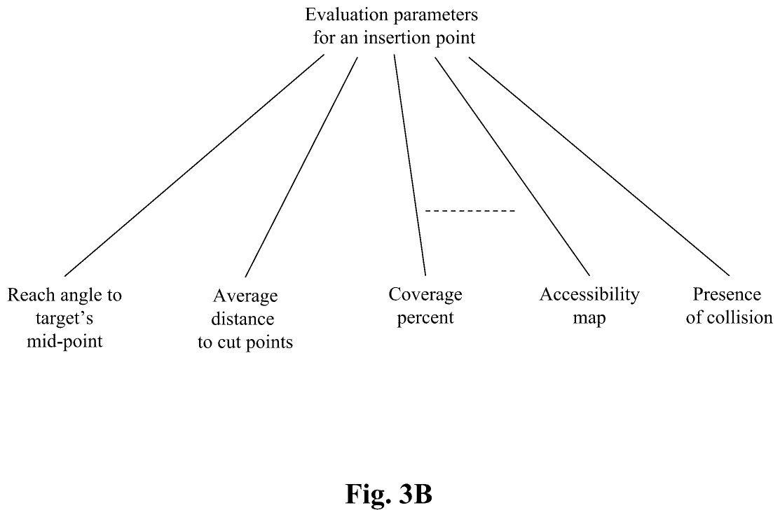

Multiple trocar locations may meet this criterion. However, to reach the cut points, the surgical instrument inserted from different trocar points may reach the cut points via different paths, some of which may be more difficult for the robot to manipulate. For instance, from a certain trocar point, the surgical instrument may collide with some non-target anatomical structures before reaching the cut points and may need to get around to avoid collision, making it more difficult to manipulate and thus less efficient. FIG. 3A illustrates that although multiple trocar points 310-1, 320-1, 330-1, and 340-1 all yield acceptable access maps 310-2, 320-2, 330-2, and 340-2 to cover preplanned cut points on organ 250, the surgical instrument inserted at different trocar points has to maneuver differently to access each of the cut points. For example, as trocar point 310-1 and 340-1 have a longer distance to some of the cut points, it is more likely that the surgical instrument may encounter other anatomical structures. In addition, from these trocar points, the tip of the surgical instrument may not be able to cut the organ at the cut point in a direction that is substantially perpendicular to the surface of the organ, making it more likely to have undesirable performance in the operation. As such, a determination is needed to select a trocar point that is appropriate or optimal given different considerations.

FIG. 3B illustrates exemplary considerations in evaluating each trocar point, in accordance with an embodiment of the present teaching. As illustrated, the considerations may include an average distance to the cut points, an angle to reach a selected cut point (e.g., a mid-point of the cut points), a percent of cut points included in an access map, . . . , whether collision exists with respect to other objects, and whether there is any collision in reaching the cut points. Additional or different evaluation criteria may also be used. As can be seen in FIG. 3A, a trocar point that is not directly above a target organ may have a higher average distance to reach the cut points and may have a more slanted angle with respect to, e.g., the average surface norm of the target organ and, hence, to the cut points. The more slanted the angle a trocar point has, the more likely that the surgical instrument inserted from the trocar point may collide with other anatomical structures along paths to reach different cut points. In addition, with a trocar point further away from a target organ, it is also more likely that its access map may not cover some of the cut points, whether it is due to collision or a too large of a distance for the surgical instrument to reach some of the cut points. Given that, not only it is important to select candidate trocar points that yield access maps that can cover all cut points but also essential to select one that is most viable in terms of risk free, efficiency, and operability.

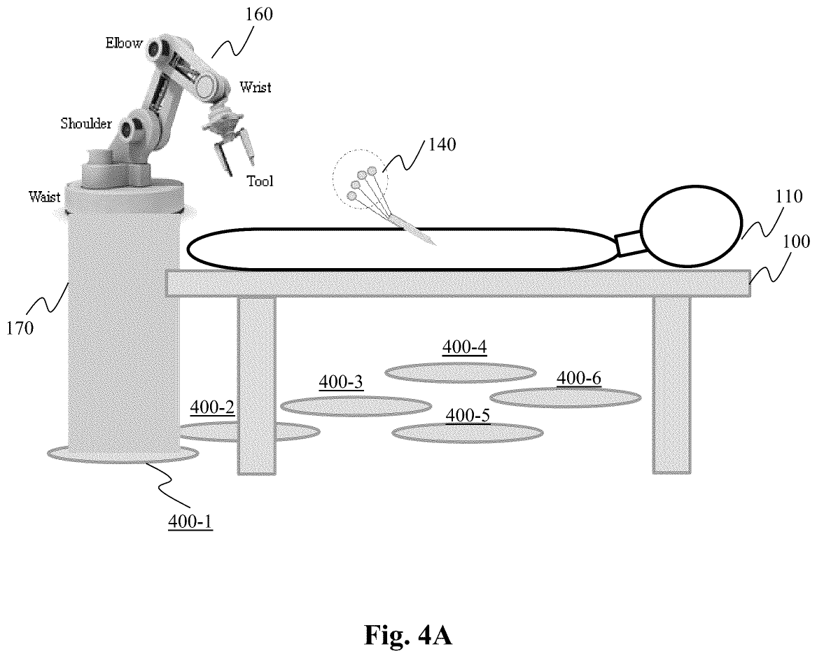

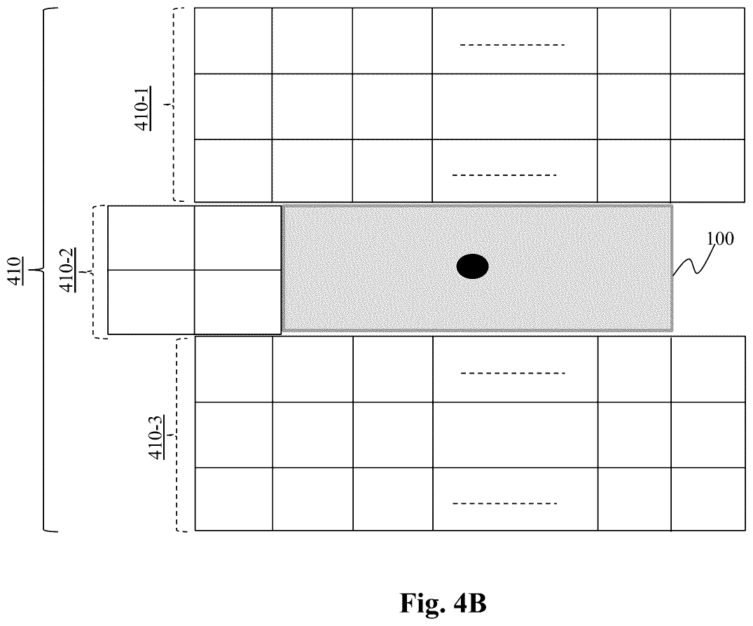

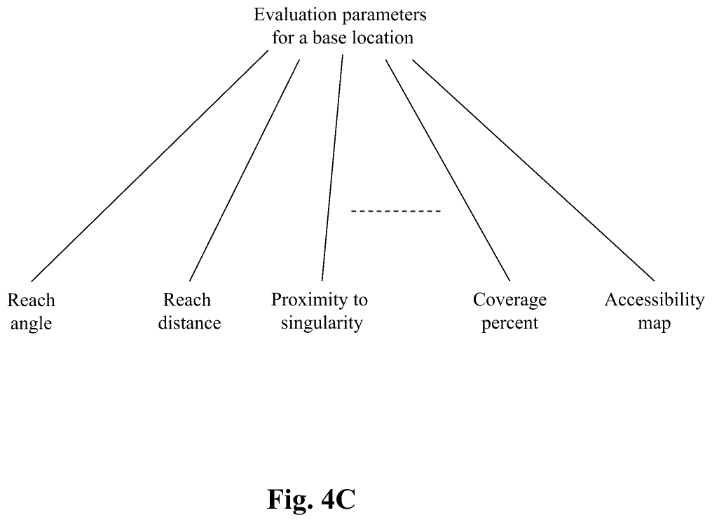

With respect to surgical room configuration, the base location of a robot is crucially important in achieving efficient operation. Although robot 160 may be placed at any one of multiple base locations in a surgery room, some base locations lead to better performance or efficiency than others. Assuming a selected trocar location, the present teaching discloses different embodiments in selecting appropriate base locations according to different considerations and further optimizing the robot base location based on manipulability of the robot 160. FIG. 4A depicts an exemplary surgical setting where a robot 160 may be placed at different base locations, in accordance with an embodiment of the present teaching. As seen, robot 160 may be positions around a surgical bed at any of multiple locations, e.g., 400-1, 400-2, 400-3, 400-4, 400-5, . . . , and 400-6. FIG. 4B shows all possible robot base locations 410 in a surgical room around different sides of a surgical bed 100. In this illustration, on each side of the surgical bed 100, the floor space may be divided into different regions, each of which is a candidate base location. As shown, along one side 410-1 of the surgical bed 100, available base locations form rows and columns of possible base locations like a grid. On the other side 410-3, there may also be rows and columns of subregions, each of which corresponds to an available base location. A third side 410-2 similarly depicts available base locations to deploy robot 160. Although base locations may be arranged as shown in FIG. 4B, other arrangements may also be possible. In some embodiments, the viable base locations may also depend on application needs. For example, if other equipment is also needed in the surgical room, the area in the surgical room for placing a robot may become more limited. There may be other limitations as well.

Although robot 160 may be able to perform an intended function from different base locations, each base location may yield different performance or results. Different criteria may be employed to evaluate different base locations with respect to intended functions for the robot. FIG. 4C presents different exemplary criteria in evaluating a robot base location, in accordance with an embodiment of the present teaching. Some examples may include the distance from the trocar to the robot base location, proximity of singularity, and a minimum distance between the robot and other robots, instruments, or obstacles. In some embodiments, other additional features may also be evaluated, including an angle between the arm and the surgical instrument, a percent of the cut points that can be reached by the surgical instrument based on the robot's access map, the kinematic feasibility of the robot arm with respect to an access map associated with each base location. It is noted that these exemplary features are provided merely for illustration rather than limitation. Other criteria may also be used. For example, when a different function is intended, a different set of criteria may be used with respect to the intended function to assess candidate base locations.

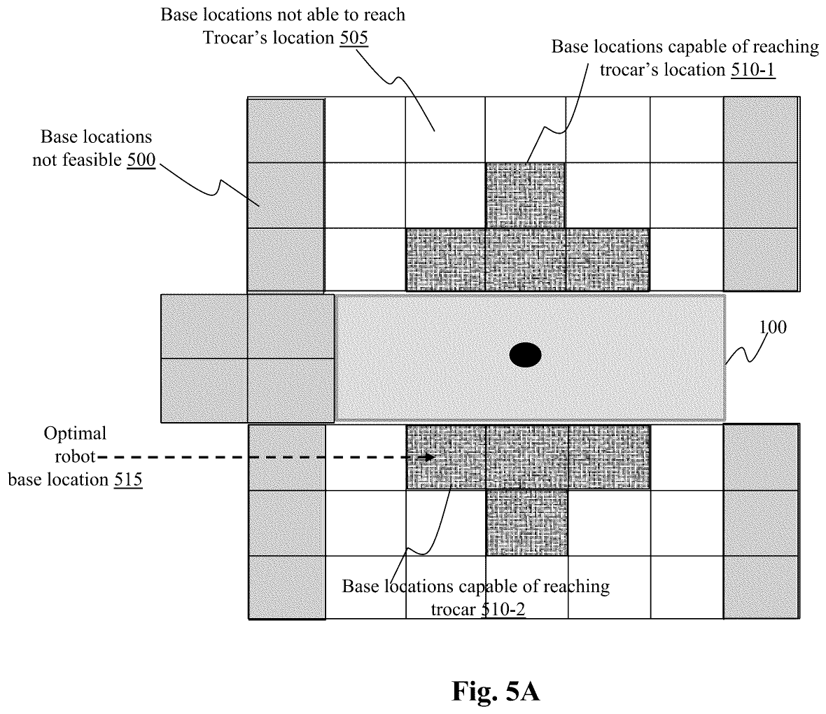

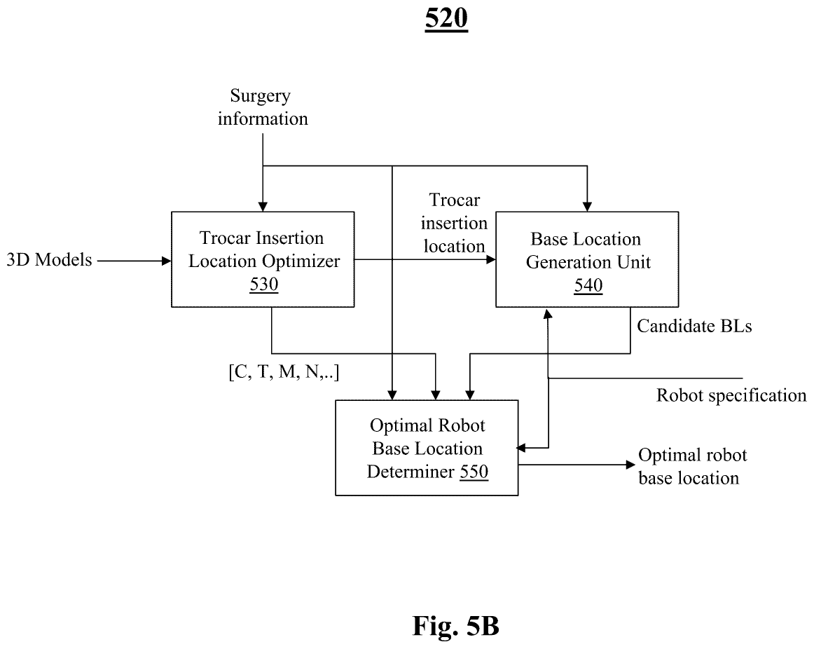

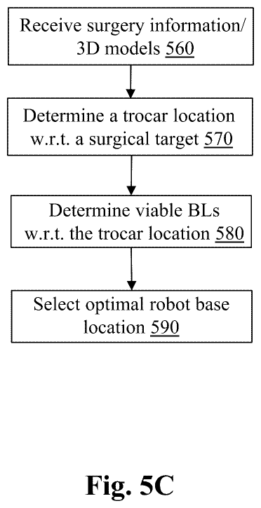

As discussed herein, the choice of a base location depends on various conditions, including a location of the trocar. For each given trocar location, there may be multiple viable robot base locations and a most suitable base location for a robot may be selected via optimization according to the present teaching. FIG. 5A shows an exemplary distribution of viable robot base locations in a surgery room and classifications thereof, in accordance with an embodiment of the present teaching. In this illustration, around a surgical bed 100, there may be a grid with all possible locations for robot placements. Some of the locations may not be viable, including some that are not feasible such as those in gray locations 500 (e.g., these locations are for placing other equipment) and some in white locations 505 from where a robot cannot reach a given trocar point. The exemplary viable base locations in FIG. 5A are those in 510, including the base locations 510-1 on one side of the surgical bed and the ones 510-2 on the opposing side of the surgical bed. The optimization as will be disclosed herein is to select an optimal robot base location 515 from viable candidate base locations in 510-1 and 510-2 with respect to some given surgery information (e.g., trocar location, target organ, etc.) and robot manipulability.

FIG. 5B depicts an exemplary high level system diagram of a robot base location optimization framework 520 based on robot manipulability with respect to given surgery information, in accordance with an embodiment of the present teaching. In this illustrated embodiment, the framework 520 comprises a trocar insertion location optimizer 530, a base location generation unit 540, and an optimal robot base location determiner 550. FIG. 5C is a flowchart of an exemplary process of the robot base location optimization framework 520, in accordance with an embodiment of the present teaching. Upon receiving, at 560, surgery information related to a surgery scheduled in a surgical room and a 3D model developed for a target organ to be operated on, the trocar insertion location optimizer 530 is provided for determining, at 570, a best trocar insertion point on a patient given the surgical information and the 3D model for the target organ to produce an optimal trocar insertion location. In some embodiments, the input surgery information may also specify a surgery area (SA) with a dimension (e.g., length of an edge of a cube encompassing the target organ) so that the trocar insertion location may be evaluated with respect to the sized surgery area.

This optimal trocar insertion location is then provided to the base location generation unit 540 to determine, at 580, candidate base locations, such as the ones in locations 510-1 and 510-2 in FIG. 5A, that are viable given the surgery area and the trocar insertion location. That is, for each robot base location generated, it is ensured that the robot is able to manipulate a surgical instrument to reach the surgery area inside of the patient via the trocar location. In some embodiments, the trocar may be defined in a trocar coordinate system. The surgery area encompassing the target organ may have a center expressed also in the trocar coordinate system. In this setting, the base location generation unit 540 may generate the candidate base locations in the trocar coordinate system so that the accessibility associated with the generated base locations may be evaluated with respect to the same coordinate system. Such candidate bas locations (BLs) by the base location generation unit 540 are then provided to the optimal robot base location determiner 550, which is to select, at 590, a best base location that represents the optimal manipulability. The selected optimal base location may be determined with respect to some criteria. As will be discussed herein, such optimality related criteria may be evaluated based on also the configuration parameters with respect to the robot at issue.

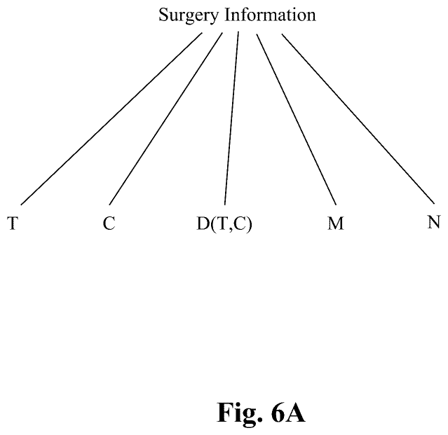

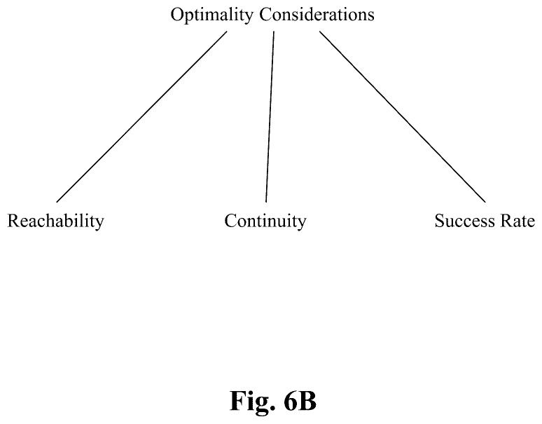

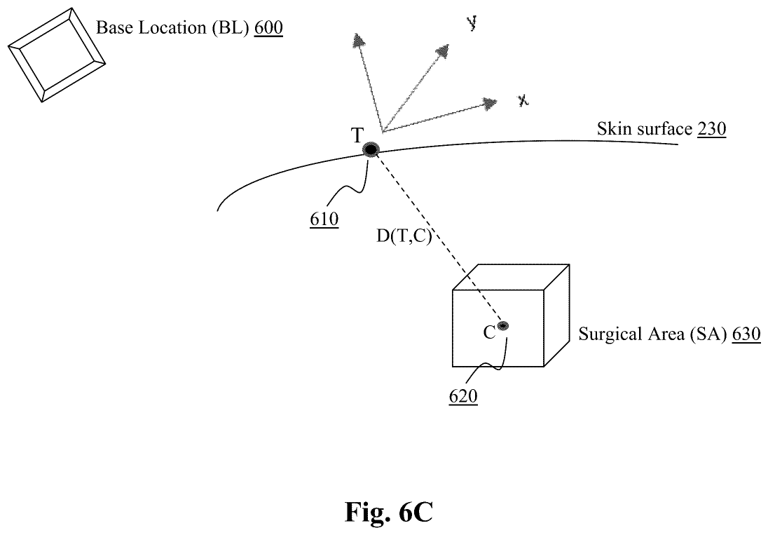

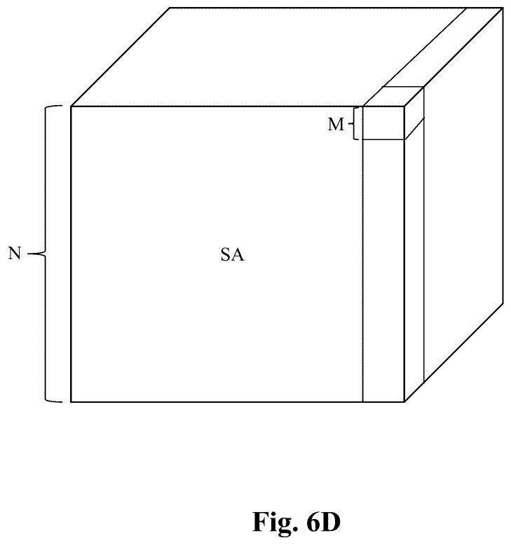

Details associated with determining an optimal trocar insertion location (by the trocar insertion location determiner 530) as well as the generation of viable candidate base locations (by the base location generation unit 540) are disclosed in the priority parent document and are referenced hereby via incorporation. The focus of the present teaching is directed to selecting an optimal base station from a given set of viable candidate base locations given a known trocar location and a known target organ location, performed by the optimal robot base location determiner 550. Details related thereto are provided with reference to FIGS. 6A-8B.

As discussed herein, viable candidate base locations of a robot deployed in a surgery room may be determined based on surgery information related to each specific surgery. FIG. 6A shows exemplary types of surgery information that determines the viability and optimality of each robot's base location, in accordance with an embodiment of the present teaching. As discussed herein, a trocar location (T) has a direct impact on the viability and optimality of a base location BL. A surgery area (SA) enclosing a target organ enclosed therein with a center C of the SA and its distance D(T,C) between center C and the trocar location T may also affect the performance of a robot at a base location. The surgery area SA corresponds to 3D volume representing a target region in a patient's body encompassing both the target organ to be operated on and its surrounding area. In some embodiments, to assess the performance of a robot standing at BL in manipulating a surgical instrument to reach different points in SA via the given trocar location T, the surgery information may also specify additional information about the SA such as a size and a resolution. For example, as shown in FIG. 6A, N may be provided to define the size of the SA (e.g., centimeter) as a cube and a resolution M defining each component cube inside SA with dimension M×M (e.g., centimeter). In some embodiments, the SA may not be a cube and in this case, the surgery information may specify (N1, N2, N3) to define the dimension of the SA. In addition, each sub-area in SA also may not be a cube and may be defined using parameters, e.g., (M1, M2, M3) to define a sub-volumetric region in SA. Although the discussion below may use cube as an illustration, it is merely for example and not intended as a limitation. With such a specified resolution, the assessment of, e.g., reachability, may be made with respect to each of such component cube in SA. This is illustrated in FIG. 6D with the dimension N for SA and M for each 3D component cube therein.

In selecting an optimal robot base location from multiple viable candidate base locations, various criteria relevant to optimality of each base location may be considered. FIG. 6B illustrates exemplary type of criteria used in assessing the optimality of a robot base location given input surgery information, in accordance with an embodiment of the present teaching. In this illustration, the optimality of a base location may be assessed in terms of reachability, continuity, . . . , and success rate. These optimality criteria as illustrated herein may be keyed on the spatial relations between the robot, the trocar location, and the location of a target area in a patient with a target organ included thereon for operation. FIG. 6C illustrates exemplary relevant spatial relations among a surgery area SA 630, a trocar location T 610, and a robot base location BL 600, in accordance with an exemplary embodiment of the present teaching. As shown herein, with respect to a candidate base location BL 600, its optimality evaluation may be related to some spatial relations, include how the BL 600 spatially relates to a trocar location T 610 on a patient's skin 230, the spatial relation and distance between T 610 and the center C 620 in SA 630.

As discussed herein, a robot at BL 600 may be assigned to handle a surgical instrument to enter the patient's body through the T 610 to perform some specified function on a targe organ enclosed in SA 630. To achieve that, one of the considerations with regard to the optimality of BL 600 is that the robot needs to be able to handle, from BL 600, the surgical instrument to reach each location within SA 630 through T 610. In some embodiments, the reachability as provided in FIG. 6B may represent a condition that requires that each point in the SA 630 is reachable from all neighboring points in SA 630. To reach a point in SA 630, the robot's joint needs to be configured accordingly to ensure that the surgical instrument can reach the point. Based on that, the optimal criterion related to continuity as provided in FIG. 6B requires that each point in SA 630 that is reachable by the robot has a continuous joint configuration from an initial joint configuration. Another consideration of the optimal assessment relates to success rate as provided in FIG. 6B, which may be defined as the percent of points in SA that are reachable by the robot from BL 600 via T 610.

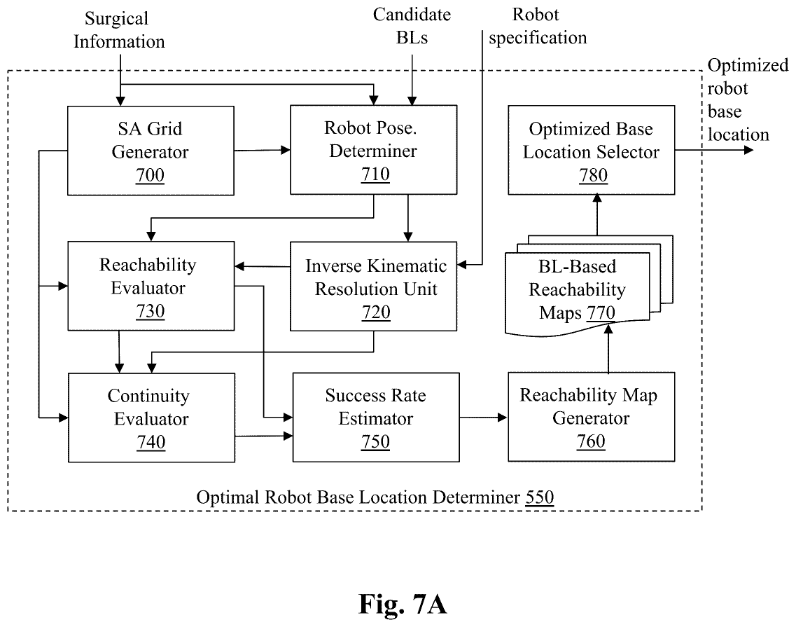

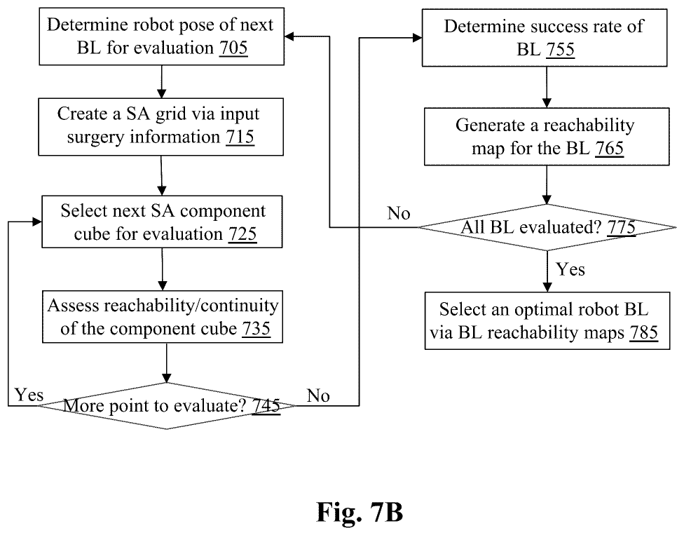

FIG. 7A depicts an exemplary high-level system diagram of the optimal robot base location determiner 550, in accordance with an embodiment of the present teaching. As discussed herein, the optimal robot base location determiner 550 takes surgery information and candidate base locations BLs as input and outputs an optimal base location which is selected from the candidate base locations. In this illustrated embodiment, the optimal robot base location determiner 550 comprises an SA grid generator 700, a robot pose determiner 710, an inverse kinematic resolution unit 720, a reachability evaluator 730, a continuity evaluator 740, a success rate estimator 750, a reachability map generator 760, and an optimized base location selector 770. As discussed herein with reference to FIG. 5A, the trocar location in each surgical setting is optimized by the trocar insertion location optimizer 530 based on surgery related information such as an SA of size N, etc. Based on the determined trocar location, the base location generation unit 540 generate candidate base locations as discussed herein with references to FIGS. 5A-5B. Given that, the optimal robot base location determiner 550 operates to select, from the input candidate base locations from 540, an optimal one based on evaluation against optimality related criteria as illustrated in FIG. 6B on reachability, continuity, and success rate.

To enable the optimality evaluation, the SA grid generator 700 is provided to create a grid in the given SA based on component cube size M in the surgery information. As discussed herein with reference to FIG. 5B and as shown in FIG. 6C, the SA is created in the trocar coordinate system (X, Y, Z). As such, the created SA grid is also in trocar coordinate system. On the other hand, the robot pose determiner 710 is provided to take each of BLs (from 540) and determines the pose of the input BL in the trocar coordinate system. With the trocar location (from the surgery information), the SA grid with component cubes therein, as well as the candidate BL all in the trocar coordinate system, the inverse kinematic resolution unit 720 is provided to make sure that the robot at the input BL can be configured to access the trocar location and the center C of SA (as provided in the surgery information). The reachability evaluator 730 is provided to assess the reachability of the robot with respect to the input BL. The continuity evaluator 740 is provided to evaluate the continuity of the robot at BL. The results of reachability and continuity evaluation are provided to the success rate estimator 750 to determine the success rate of the BL. The evaluation results in reachability, continuity, and success rate are then used by the reachability map generator 760 to create a reachability map for the input BL, which is then stored in BL-based reachability maps storage 770. This is performed for each of the candidate BLs output from the base location generation unit 540 (FIG. 5A) so that for each candidate BL, a BL-based reachability map is stored in 770. When all candidate BLs are evaluated as discussed herein, the optimized base location selector 780 selects, based on the archived reachability maps, one base location that is the best in terms of reachability, continuity, and success rate.

In some embodiments, a reachability map for a candidate BL may include a 3D array and some overall metrics. The 3D array in the reachability map may have the same structure as that of the SA as shown in FIG. 6D, with each element therein corresponding to a component cube in the SA in FIG. 6D and stores evaluation metrics associated with the component cube to indicate, e.g., the reachability and continuity of the component cube. The overall metrics associated with each BL may represent the success rates in terms of respectively the overall reachability and continuity of the BL determined based on the percent of the component cubes that are reachable and continuous. That is, the BL-based reachability map for each candidate BL evaluates not only the performance with respect to individual component cubes inside the SA but also the overall performance in terms of statistics. Based on such characterization of each of the candidate BLs, the optimal BL may be selected according to some criterion specified based on application needs.

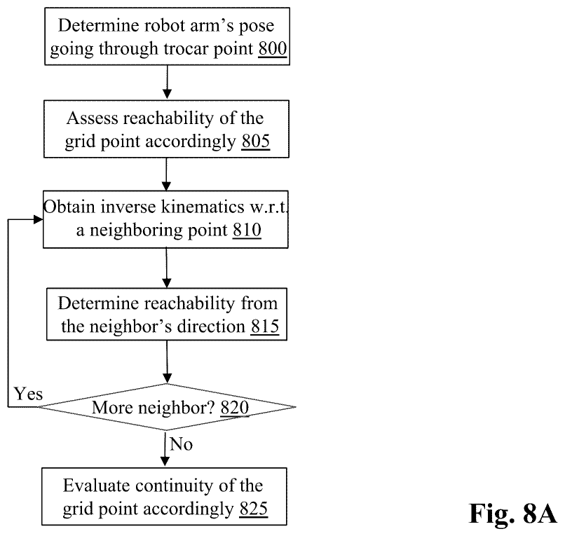

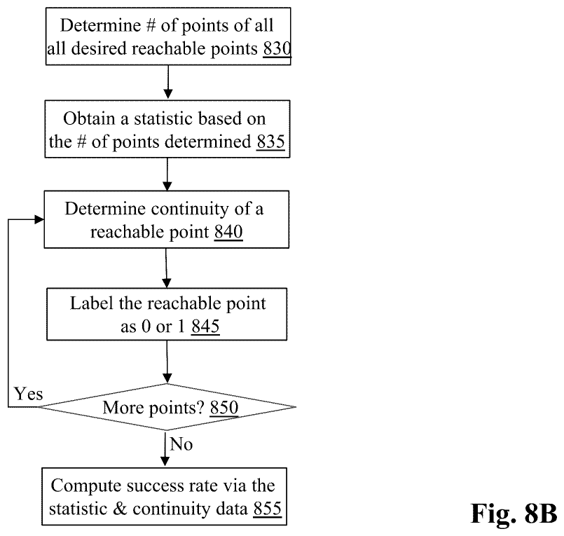

FIG. 7B is a flowchart of an exemplary process of the optimal robot base location determiner, in accordance with an embodiment of the present teaching. As discussed herein, in operation, when candidate BLs are received from 540, each candidate BL is evaluated separately. To do so, the 3D pose of a candidate BL is first determined, at 705, to facilitate the evaluation of the BL. To assess the reachability and continuity of each point in a SA associated with the current BL to be evaluated, an SA grid is created, at 715, based on the input surgery information, including C, N, and M, where the size of the SA is N×N with each component cube therein with a dimension of M×M. At 725, a next component cube in the SA grid is selected for assessing, at 735, the reachability and continuity related to the component cube. Each of the component cube is individually evaluated until all component cubes in the SA grid have been evaluated, as determined at 745. Details relating to assessing the reachability/continuity of each component cube are provided in FIG. 8A.

Once all component cubes in an SA grid are evaluated, the process proceeds to step 755 to determine the overall success rate of the current BL based on the evaluation result with respect to each of the component cubes on the percent of the component cubes in the SA grid that are reachable by a robot from the BL and/or the percent of the component cubes that have continuity. Details related to the overall success rate of a SA grid are provided with reference to FIG. 8B. Based on the SA grid with individually evaluated component cubes as well as the overall success rate for the SA, a reachability map is created, at 765, for the current BL and may be stored in 770 under the current BL. If there are more candidate BL to be evaluated, determined at 775, the process returns to steps 705-765, as discussed herein. If all candidate BLs have been evaluated, their corresponding reachability maps stored in 770 are then used by the optimized base location selector 780 to select, at 785, an optimal robot base location.

FIG. 8A is a flowchart of an exemplary process of assessing reachability/continuity of each component cube in a surgery area SA, in accordance with an embodiment of the present teaching. As discussed herein, the reachability evaluation (performed by the reachability evaluator 739 with respect to a component cube is to determine whether the robot arm may be configured in such a way that the surgical instrument manipulated by the robot arm can reach the component cube. In addition, the continuity of each component cube in the SA (by the continuity evaluator 740) is to see whether the component cube may be reached from its neighboring component cubes. As such, for each component cube in an SA grid, the pose of a robot arm's pose going through a given trocar location may be first determined at 800. In some embodiments, as the trocar location is represented in a trocar coordinate system, the robot arm's pose may also be represented in the same trocar coordinate system.

From the robot arm's pose at the trocar location, the reachability evaluator 730 assesses, at 805, whether the current component cube is reachable by determining whether the robot arm parameters may be configured to reach the component cube from the trocar location. In some embodiments, the reachability of the component cube may be recorded (either reachable or non-reachable). The continuity evaluation is performed with respect to all neighbors of the component cube in the SA grid. In some embodiments, for each component cube in the 3D SA, there may be 6 neighbors, including up, down, left, right, front, and back neighboring component cubes. In some embodiments, there may be more neighbors including those component cubes in the SA that are diagonally connected. To assess the continuity of a component cube, for each of its neighbor component cube, inverse kinematics of a robot arm may be obtained, at 810, with respect to the neighbor and then determine, at 815, whether the robot arm (currently inversely configured via inverse kinematics) may reach the component cube from the neighbor's position. The inverse reachability is evaluated with respect to all neighbor component cubes, as controlled at 820. If the component cube currently being evaluated on continuity can be reached rom all of its neighbor component cube positions, then the component cube may be characterized with possessing continuity.

FIG. 8B is a flowchart of an exemplary process of assessing the success rate with respect to each base location performed by the success rate estimator 750, in accordance with an embodiment of the present teaching. As discussed herein, when all the component cubes in a SA grid have been evaluated in terms of reachability and continuity, the success rate estimator 750 is invoked to estimate the overall success rate of the SA grid based on the SA grid with each component cube recorded as to its reachability and continuity. At 830, the total number of component cubes in SA grid that can be reached by a robot at a candidate BL is determined and accordingly a statistic, e.g., a percent of the component cubes that are reachable, may be obtained at 835, to represent the overall reachability. In some embodiments, the overall success rate with respect to continuity, the assessment may be made with respect to, e.g., reachable component cubes in SA grid. Based on the continuity with respect to each reachable point, assessed by the continuity evaluator 740, the success rate estimator 750 labels each component cube, at 845, according to its continuity evaluation, e.g., label 1 if it has continuity or 0 if not. The process of labeling each component cubes based on its continuity evaluation continues until, determined at 850, all reachable component cubes have been labels. Then the success rate is computed, at 855, based on the statistic related to reachability and the continuity labels. As discussed herein, the assessments in terms of reachability, continuity, and success rate are then used by the optimized base location selector 780 to choose one of the candidate BLs as the optimal robot base location.

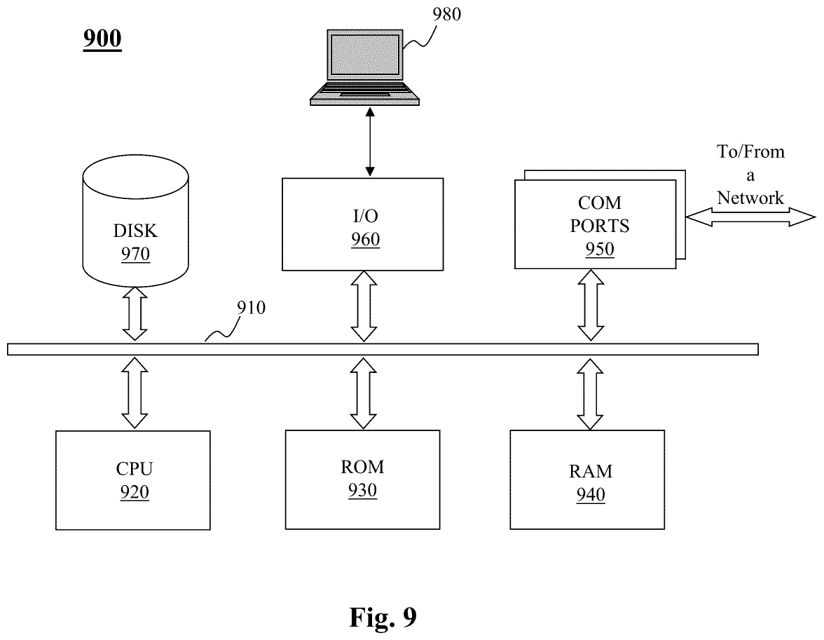

FIG. 9 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special-purpose computer. Both can be used to implement a specialized system for the present teaching. This computer 900 may be used to implement any component or aspect of the framework as disclosed herein. For example, the information analytical and management method and system as disclosed herein may be implemented on a computer such as computer 900, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 900, for example, includes COM ports 950 connected to and from a network connected thereto to facilitate data communications. Computer 900 also includes a central processing unit (CPU) 920, in the form of one or more processors, for executing program instructions. The exemplary computer platform includes an internal communication bus 910, program storage and data storage of different forms (e.g., disk 970, read only memory (ROM) 930, or random-access memory (RAM) 940), for various data files to be processed and/or communicated by computer 900, as well as possibly program instructions to be executed by CPU 920. Computer 900 also includes an I/O component 960, supporting input/output flows between the computer and other components therein such as user interface elements 980. Computer 900 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine-readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable the loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or a physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as the main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, a hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method, comprising:
receiving surgery information related to a surgery on a target organ of a patient in a surgery space in which a robot is used to manipulate a surgical instrument to perform an operation directed to the target organ;
determining, based on the surgery information, a trocar location on the patient for inserting the surgical instrument to reach the target organ;
generating, according to the trocar location and the target organ, candidate base locations within the surgery space, each of which corresponds to a location to deploy the robot for manipulating the surgical instrument to reach the target organ through the trocar location;
evaluating each of the candidate base locations according to predetermined criteria indicative of the robot's manipulability of the surgical instrument from the candidate base location with respect to the target organ; and
selecting, based on the evaluation result on each of the candidate base locations, an optimal base location from the candidate base locations.

2. The method of claim 1, wherein the surgery information includes:
a point near the target organ;
a distance between the point and the trocar location;
dimensional parameters defining a surgery area centered at the point, which represents a three-dimensional (3D) region around the target organ and encloses the target organ.

3. The method of claim 2, wherein the dimensional parameters include:
a first set of parameters defining the dimension of the surgery area; and
a second set of parameters defining the dimension of each of multiple sub-areas obtained by dividing the surgery area into a grid of the multiple sub-areas.

4. The method of claim 3, wherein the surgery area grid with multiple sub-areas is obtained to facilitate the evaluation of each of the candidate base locations in terms of whether the robot at the candidate base location is able to reach each of the multiple sub-areas in the surgery area.

5. The method of claim 4, wherein the predetermined criteria used to evaluate each of the candidate base locations include:
reachability of each of the sub-areas in the surgery area grid;
continuity of each of the sub-areas in the surgery area grid; and
overall success rate with respect to the surgery area determined based on the reachability and continuity associated with each of the sub-areas of the surgery area.

6. The method of claim 5, wherein
the reachability with respect to a sub-area is defined to indicate whether the surgical instrument manipulated by the robot at a candidate base location is able to reach the sub-area; and
the continuity associated with a sub-area is defined to indicate whether the surgical instrument manipulated by the robot at a candidate base location is able to reach the sub-area from adjacent sub-areas.

7. The method of claim 6, wherein the step of evaluating each of the candidate base locations comprises:
with respect to each of the sub-areas in the surgery area grid,
determining reachability of the sub-area, and
assessing continuity of the sub-area;
assessing success rate associated with the candidate base location based on the reachability and continuity associated with each of the sub-areas in the surgery area grid.

8. A machine-readable and non-transitory medium having information recorded thereon, wherein the information, when read by the machine, causes the machine to perform the following steps:
receiving surgery information related to a surgery on a target organ of a patient in a surgery space in which a robot is used to manipulate a surgical instrument to perform an operation directed to the target organ;
determining, based on the surgery information, a trocar location on the patient for inserting the surgical instrument to reach the target organ;
generating, according to the trocar location and the target organ, candidate base locations within the surgery space, each of which corresponds to a location to deploy the robot for manipulating the surgical instrument to reach the target organ through the trocar location;
evaluating each of the candidate base locations according to predetermined criteria indicative of the robot's manipulability of the surgical instrument from the candidate base location with respect to the target organ; and
selecting, based on the evaluation result on each of the candidate base locations, an optimal base location from the candidate base locations.

9. The medium of claim 8, wherein the surgery information includes:
a point near the target organ;
a distance between the point and the trocar location;
dimensional parameters defining a surgery area centered at the point, which represents a three-dimensional (3D) region around the target organ and encloses the target organ.

10. The medium of claim 9, wherein the dimensional parameters include:
a first set of parameters defining the dimension of the surgery area; and
a second set of parameters defining the dimension of each of multiple sub-areas obtained by dividing the surgery area into a grid of the multiple sub-areas.

11. The medium of claim 10, wherein the surgery area grid with multiple sub-areas is obtained to facilitate the evaluation of each of the candidate base locations in terms of whether the robot at the candidate base location is able to reach each of the multiple sub-areas in the surgery area.

12. The medium of claim 11, wherein the predetermined criteria used to evaluate each of the candidate base locations include:
reachability of each of the sub-areas in the surgery area grid;
continuity of each of the sub-areas in the surgery area grid; and
overall success rate with respect to the surgery area determined based on the reachability and continuity associated with each of the sub-areas of the surgery area.

13. The medium of claim 12, wherein
the reachability with respect to a sub-area is defined to indicate whether the surgical instrument manipulated by the robot at a candidate base location is able to reach the sub-area; and
the continuity associated with a sub-area is defined to indicate whether the surgical instrument manipulated by the robot at a candidate base location is able to reach the sub-area from adjacent sub-areas.

14. The medium of claim 13, wherein the step of evaluating each of the candidate base locations comprises:
with respect to each of the sub-areas in the surgery area grid,
determining reachability of the sub-area, and
assessing continuity of the sub-area;
assessing success rate associated with the candidate base location based on the reachability and continuity associated with each of the sub-areas in the surgery area grid.

15. A system, comprising:
a trocar insertion location optimizer implemented by a processor and configured for
receiving surgery information related to a surgery on a target organ of a patient in a surgery space in which a robot is used to manipulate a surgical instrument to perform an operation directed to the target organ, and
determining, based on the surgery information, a trocar location on the patient for inserting the surgical instrument to reach the target organ;
a base location generator implemented by a processor and configured for generating, according to the trocar location and the target organ, candidate base locations within the surgery space, each of which corresponds to a location to deploy the robot for manipulating the surgical instrument to reach the target organ through the trocar location; and
an optimal robot base location determiner implemented by a processor and configured for:
evaluating each of the candidate base locations according to predetermined criteria indicative of the robot's manipulability of the surgical instrument from the candidate base location with respect to the target organ, and
selecting, based on the evaluation result on each of the candidate base locations, an optimal base location from the candidate base locations.

16. The system of claim 15, wherein the surgery information includes:
a point near the target organ;
a distance between the point and the trocar location;
dimensional parameters defining a surgery area centered at the point, which represents a three-dimensional (3D) region around the target organ and encloses the target organ.

17. The system of claim 16, wherein the dimensional parameters include:
a first set of parameters defining the dimension of the surgery area; and
a second set of parameters defining the dimension of each of multiple sub-areas obtained by dividing the surgery area into a grid of the multiple sub-areas.

18. The system of claim 17, wherein the surgery area grid with multiple sub-areas is obtained to facilitate the evaluation of each of the candidate base locations in terms of whether the robot at the candidate base location is able to reach each of the multiple sub-areas in the surgery area.

19. The system of claim 18, wherein the predetermined criteria used to evaluate each of the candidate base locations include:
reachability of each of the sub-areas in the surgery area grid;
continuity of each of the sub-areas in the surgery area grid; and
overall success rate with respect to the surgery area determined based on the reachability and continuity associated with each of the sub-areas of the surgery area.

20. The system of claim 19, wherein
the reachability with respect to a sub-area is defined to indicate whether the surgical instrument manipulated by the robot at a candidate base location is able to reach the sub-area; and
the continuity associated with a sub-area is defined to indicate whether the surgical instrument manipulated by the robot at a candidate base location is able to reach the sub-area from adjacent sub-areas.

21. The system of claim 20, wherein the step of evaluating each of the candidate base locations comprises:
with respect to each of the sub-areas in the surgery area grid,
determining reachability of the sub-area, and
assessing continuity of the sub-area;
assessing success rate associated with the candidate base location based on the reachability and continuity associated with each of the sub-areas in the surgery area grid.
